(19) United States
(12) Patent Application Publication
(10) Pub. No.: US 2021/0386305 A1
(43) Pub. Date: Dec. 16, 2021

(54) APPARATUS AND METHOD FOR ESTIMATING BLOOD PRESSURE

(71) Applicant: SAMSUNG ELECTRONICS CO., LTD., Suwon-si (KR)

(72) Inventors: YONG JOO KWON, Yongin-si (KR); UI KUN KWON, Hwaseong-si (KR); JAE MIN KANG, Seoul (KR); YOUN HO KIM, Hwaseong-si (KR)

(73) Assignee: SAMSUNG ELECTRONICS CO., LTD., Suwon-si (KR)

(21) Appl. No.: 17/097,077

(22) Filed: Nov. 13, 2020

(30) Foreign Application Priority Data
Jun. 15, 2020 (KR) 10-2020-0072333

(51) Int. Cl. A61B 5/021; A61B 90/00

(52) U.S. Cl. CPC A61B 5/02108; A61B 2090/064; A61B 90/06; A61B 5/02141

(57) ABSTRACT

Provided is an apparatus for estimating blood pressure. The apparatus for estimating blood pressure according to an embodiment of the disclosure includes: a pulse wave measurer configured to measure a pulse wave signal from a user; and a processor configured to detect peaks and valleys from the pulse wave signal, to obtain first differential values, corresponding to the detected peaks, and second differential values, corresponding to the detected valleys, from a second-order differential signal of the pulse wave signal, and to estimate blood pressure based on the obtained first differential values and second differential values.

### CROSS-REFERENCE TO RELATED APPLICATION(S)

This application claims priority from Korean Patent Application No. 10-2020-0072333, filed on Jun. 15, 2020, in the Korean Intellectual Property Office, the entire disclosure of which is herein incorporated by reference for all purposes.

### BACKGROUND

### 1. Field

The disclosure relates to technology for estimating blood pressure, and more particularly to technology for estimating systolic blood pressure and diastolic blood pressure by second-order differentiation of pulse waves.

### 2. Description of the Related Art

With the aging population, soaring medical costs, and a lack of medical personnel for specialized medical services, research is being actively conducted on information technology (IT)-medical convergence technologies, in which IT and medical technology are combined. Particularly, monitoring of the health condition of a human body is not limited to medical institutions, but is expanding to mobile healthcare fields that may monitor a user's health condition anywhere and anytime in daily life, e.g., at home or office. Typical examples of bio-signals, which indicate the health condition of individuals, include an electrocardiography (ECG) signal, a photoplethysmography (PPG) signal, an electromyography (EMG) signal, and the like, and various bio-signal sensors have been developed to measure these signals in daily life. Particularly, a PPG sensor may estimate blood pressure of a human body by analyzing a shape of pulse waves which reflect cardiovascular status and the like.

According to studies on the PPG signal, the entire PPG signal is a superposition of propagation waves departing from the heart and moving toward the distal portions of the body, and reflection waves returning from the distal portions. Further, it has been known that information for estimating blood pressure may be obtained by extracting various features associated with the propagation waves or the reflection waves.

### SUMMARY

In accordance with an aspect of an example embodiment, there is provided an apparatus for estimating blood pressure, the apparatus including: a pulse wave measurer configured to measure a pulse wave signal from an object of a user; and a processor configured to detect peaks and valleys from the pulse wave signal, to obtain first differential values, corresponding to the detected peaks, and second differential values, corresponding to the detected valleys, from a second-order differential signal of the pulse wave signal, and to estimate blood pressure based on the obtained first differential values and second differential values.

The processor may obtain pressure exerted between the object and the pulse wave measurer during measurement of the pulse wave signal.

The apparatus for estimating blood pressure may further include a force sensor configured to measure a force exerted between the pulse wave measurer and the object, wherein the processor may obtain the pressure based on the measured force.

The apparatus for estimating blood pressure may further include a contact area sensor configured to measure a contact area between the object and the pulse wave measurer, wherein the processor may obtain the pressure based on the measured force and the contact area.

The processor may estimate the blood pressure based on a first pressure at a position of a minimum value among the first differential values, and a second pressure at a position of a maximum value among the second differential values.

Based on a relationship between a waveform of the pulse wave signal and the blood pressure, the processor may determine one of the first pressure and the second pressure to be systolic blood pressure and determine the other one of the first pressure and the second pressure to be diastolic blood pressure.

Based on a proportional relationship between the waveform of the pulse wave signal and the blood pressure, the processor may determine the first pressure to be the systolic blood pressure and determine the second pressure to be the diastolic blood pressure.

Based on an inversely proportional relationship between the waveform of the pulse wave signal and the blood pressure, the processor may determine the first pressure to be the diastolic blood pressure and determine the second pressure to be the systolic blood pressure.

Based on at least one of the waveform of the pulse wave signal and a method of measuring the pulse wave signal, the processor may determine the relationship between the waveform of the pulse wave signal and the blood pressure.

The pulse wave measurer may include at least one of a cuff device and a photoplethysmography (PPG) sensor.

In addition, the apparatus for estimating blood pressure may further include an output interface configured to output guide information for guiding a contact state between the object and the pulse wave measurer.

The processor may perform filtering of the measured pulse wave signal.

In accordance with an aspect of an example embodiment, there is provided a method of estimating blood pressure, the method including: measuring, by using a pulse wave measurer, a pulse wave signal from an object of a user; detecting peaks and valleys from the pulse wave signal; obtaining first differential values, corresponding to the detected peaks, and second differential values, corresponding to the detected valleys, from a second-order differential signal of the pulse wave signal; and estimating blood pressure based on the obtained first differential values and second differential values.

The method of estimating blood pressure may further include obtaining pressure exerted between the object and the pulse wave measurer during measurement of the pulse wave signal.

The obtaining the pressure may include measuring a force exerted between the pulse wave measurer and the object, and obtaining the pressure based on the measured force.

The obtaining the pressure may further include measuring a contact area between the object and the pulse wave measurer, and obtaining the pressure based on the measured force and the contact area.

The estimating the blood pressure may include estimating the blood pressure based on a first pressure at a position of a minimum value among the first differential values, and a second pressure at a position of a maximum value among the second differential values.

The estimating the blood pressure may include, based on a relationship between a waveform of the pulse wave signal and blood pressure, determining one of the first pressure and the second pressure to be systolic blood pressure and determine the other one of the first pressure and the second pressure to be diastolic blood pressure.

The estimating the blood pressure may include, based on a proportional relationship between the waveform of the pulse wave signal and the blood pressure, determining the first pressure to be the systolic blood pressure and determining the second pressure to be the diastolic blood pressure.

The estimating the blood pressure may include, based on an inversely proportional relationship between the waveform of the pulse wave signal and the blood pressure, determining the first pressure to be the diastolic blood pressure and determining the second pressure to be the systolic blood pressure.

The method of estimating blood pressure may further include outputting guide information for guiding a contact state between the object and the pulse wave measurer.

In accordance with an aspect of an example embodiment, there is provided an apparatus for estimating blood pressure, the apparatus including: a communication interface configured to receive a pulse wave signal from an external device; and a processor configured to detect peaks and valleys from the received pulse wave signal, to obtain first differential values, corresponding to the detected peaks, and second differential values, corresponding to the detected valleys, from a second-order differential signal of the pulse wave signal, and to estimate blood pressure based on the obtained first differential values and second differential values.

### BRIEF DESCRIPTION OF THE DRAWINGS

The above and other aspects and features of the disclosure will become more apparent by describing in detail example embodiments thereof with reference to the attached drawings.

FIG. 1 is a block diagram illustrating an apparatus for estimating blood pressure according to an embodiment of the disclosure.

FIGS. 2A and 2B are block diagrams illustrating an apparatus for estimating blood pressure according to other embodiments of the disclosure.

FIGS. 3A to 3C are diagrams illustrating an example of estimating systolic blood pressure and diastolic blood pressure according to an embodiment of the disclosure.

FIG. 4 is a flowchart illustrating a method of estimating blood pressure according to an embodiment of the disclosure.

FIG. 5 is a diagram illustrating a wearable device according to an embodiment of the disclosure.

FIG. 6 is a diagram illustrating a smart device according to an embodiment of the disclosure.

FIG. 7 is a cuff-type blood pressure measurer according to an embodiment of the disclosure.

### DETAILED DESCRIPTION

Details of example embodiments are included in the following detailed description and drawings. Advantages and features of the disclosure, and a method of achieving the same will be more clearly understood from the following embodiments described in detail with reference to the accompanying drawings. Throughout the drawings and the detailed description, unless otherwise described, the same drawing reference numerals will be understood to refer to the same elements, features, and structures. The relative size and depiction of these elements may be exaggerated for clarity, illustration, and convenience.

It will be understood that, although the terms first, second, etc. may be used herein to describe various elements, these elements should not be limited by these terms. These terms are only used to distinguish one element from another. Any references to singular may include plural unless expressly stated otherwise. In addition, unless explicitly described to the contrary, an expression such as “comprising” or “including” will be understood to imply the inclusion of stated elements but not the exclusion of any other elements. Also, the terms, such as ‘part’ or ‘module’, etc., should be understood as a unit that performs at least one function or operation and that may be embodied as hardware, software, or a combination thereof.

Hereinafter, embodiments of an apparatus and method for estimating blood pressure will be described in detail with reference to the accompanying drawings.

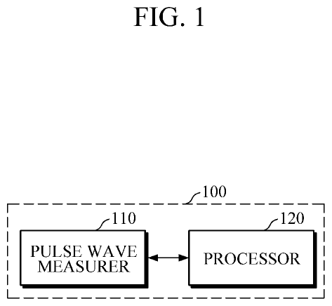

FIG. 1 is a block diagram illustrating an apparatus for estimating blood pressure according to an embodiment of the disclosure. Various embodiments of an apparatus 100 for estimating blood pressure may be embedded in a terminal, such as a smartphone, a tablet personal computer (PC), a desktop computer, a laptop computer, a wearable device, and the like. Examples of the wearable device may include a wristwatch-type wearable device, a bracelet-type wearable device, a wristband-type wearable device, a ring-type wearable device, a glasses-type wearable device, a headband-type wearable device, or the like, but the wearable device is not limited thereto, and may be embedded in a cuff-type blood pressure measuring device, or may be embedded in hardware manufactured in various shapes for use in medical institutions.

Referring to FIG. 1, the apparatus 100 for estimating blood pressure includes a pulse wave measurer 110 and a processor 120.

The pulse wave measurer 110 may measure an oscillometric pulse wave signal from a user's object.

For example, the pulse wave measurer 110 may include a photoplethysmography (PPG) sensor for measuring a PPG signal from the object, or a cuff device which acquires an oscillometric pulse wave signal from the user's upper arm. The object may be an area on a wrist that is adjacent to a radial artery, an upper portion of the wrist where veins or capillaries are located, or distal portions of the body, such as fingers, toes, and the like where blood vessels are densely located.

The PPG sensor may include a light source for emitting light onto the object and a detector for measuring the PPG signal by detecting light emanating from the object when light, emitted by the light source, is scattered or reflected from body tissue of the object. In this case, the light source may include at least one of a light emitting diode (LED), a laser diode (LD), and a phosphor, but is not limited thereto. The detector may include a photo diode.

The processor 120 may be electrically or mechanically connected to the pulse wave measurer 110, or may be connected by wire or wirelessly to the pulse wave measurer 110, depending on the pulse wave measurer 110. Upon receiving a request for estimating blood pressure, the processor 120 may control the pulse wave measurer 110, and may receive the oscillometric pulse wave signal from the pulse wave measurer 110.

Upon receiving the pulse wave signal from the pulse wave measurer 110, the processor 120 may perform preprocessing, such as filtering for removing noise, amplifying the pulse wave signal, converting the signal into a digital signal, and the like. For example, the processor 120 may remove noise from the pulse wave signal, received from the pulse wave measurer 110, by performing band-pass filtering between 0.4 Hz to 10 Hz by using a band-pass filter. Further, the processor 120 may correct the pulse wave signal by reconstructing the pulse wave signal using Fast Fourier Transform (FFT). However, the processor 120 is not limited thereto, and may perform various other preprocessing operations according to various measurement environments, such as computing performance or measuring accuracy of a device, purpose of blood pressure estimation, a measured portion of the user, temperature and humidity of the object, temperature of the pulse wave measurer, and the like.

The processor 120 may detect peaks and valleys from the pulse wave signal received from the pulse wave measurer 110. Further, the processor 120 may derive a differential signal by performing second-order differentiation of the pulse wave signal, and may estimate blood pressure based on the detected peaks and valleys.

For example, the processor 120 may obtain first differential values at the peaks and second differential values at the valleys from the second-order differential signal, and may estimate systolic blood pressure or diastolic blood pressure based on a first pressure between an object and the pulse wave measurer 110, the first pressure corresponding to a position of a minimum value among the first differential values, and a second pressure between an object and the pulse wave measurer 110, the second pressure corresponding to a position of a maximum value among the second differential values.

For example, if a relationship between blood pressure and a waveform of the pulse wave signal is a proportional relationship, the processor 120 may determine the first pressure to be systolic blood pressure and the second pressure to be diastolic blood pressure. By contrast, if a relationship between blood pressure and a waveform of the pulse wave signal is an inversely proportional relationship, the processor 120 may determine the first pressure to be diastolic blood pressure and the second pressure to be systolic blood pressure.

In this case, the relationship between blood pressure and the pulse wave signal may be determined according to a method of measuring pulse waves, a shape of the waveform of the pulse wave signal, and the like. For example, if the pulse wave measurer 110 measures pulse waves from the user's upper arm or using a finger cuff, the relationship between blood pressure and the pulse wave signal may be determined to be a proportional relationship, and if the pulse wave measurer 110 measures a PPG signal, the relationship between blood pressure and the pulse wave signal may be determined to be an inversely proportional relationship. In another example, upon analyzing a waveform of the pulse wave signal measured by the pulse wave measurer 110, the processor 120 may determine that there is a proportional relationship between blood pressure and the pulse wave signal if the waveform has a positive maximum slope, and may determine that there is an inversely proportional relationship between blood pressure and the pulse wave signal if the waveform has a negative maximum slope. In this case, the processor 120 may obtain a first-order differential signal of the pulse wave signal, and if a value at a point, where an absolute value of the first-order differential value is maximum, is a positive value, the processor 120 may determine the maximum slope to be positive; and if a value at a point, where an absolute value of the first-order differential value is maximum, is a negative value, the processor 120 may determine the maximum slope to be negative.

In addition, the processor 120 may obtain pressure exerted between the user's object and the pulse wave measurer 110 while the pulse wave signal is measured. For example, if the pulse wave measurer 110 is a cuff device for measuring oscillometric pulse waves from the user's upper arm, the processor 120 may obtain cuff pressure applied by the pulse wave measurer 110 to the user's upper arm. In another example, if the pulse wave measurer 110 is a PPG sensor for measuring oscillometric pulse waves from the user's finger or wrist, the processor 120 may obtain pressure between the object and the pulse wave measurer 110 by measuring a force or pressure applied to the PPG sensor when the finger or the wrist comes into contact with the PPG sensor and presses the sensor.

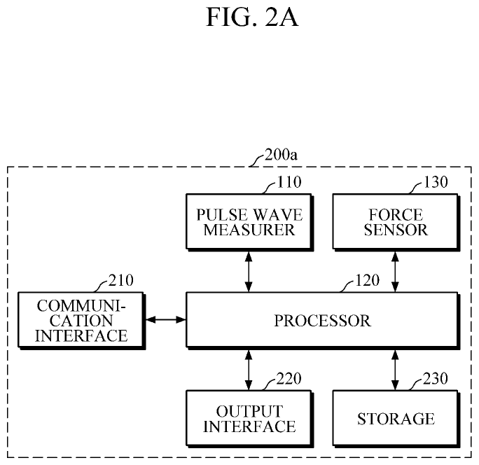

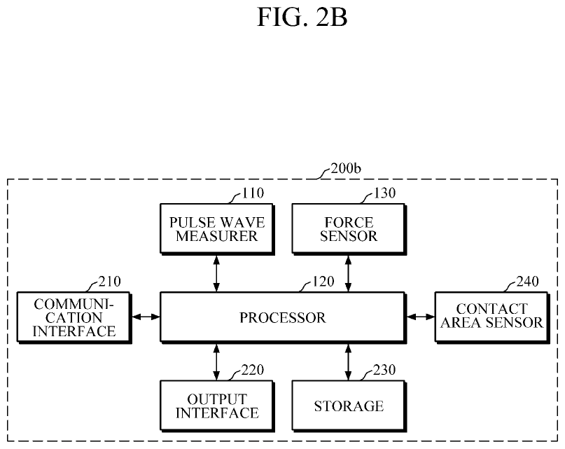

FIGS. 2A and 2B are block diagrams illustrating an apparatus for estimating blood pressure according to other embodiments of the disclosure.

Referring to FIGS. 2A and 2B, apparatuses 200a and 200b for estimating blood pressure may include the pulse wave measurer 110, the processor 120, a force sensor 130, a communication interface 210, an output interface 220, and a storage 230.

As described above, the pulse wave measurer 110 may include a PPG sensor or a cuff device which may measure an oscillometric pulse wave signal. In an example embodiment, the pulse wave measurer 110 may be omitted as will be described below.

When the user's object comes into contact with the pulse wave measurer 110 and increases or decreases a pressing force on the pulse wave measurer 110, the force sensor 130 may measure a force applied by the pulse wave measurer 110 to the object. The force sensor 130 may include a strain gauge, and may measure a user's pressing force on the pulse wave measurer 110.

The processor 120 may obtain pressure exerted between the object and the pulse wave measurer 110 based on the force measured by the force sensor 130. For example, the processor 120 may obtain pressure based on an area of a contact surface between the object and the pulse wave measurer 110, and the force measured by the force sensor 130. In another example, the processor 120 may obtain contact pressure from the contact force by applying a conversion model which defines a correlation between the contact force and the contact pressure.

In addition, referring to FIG. 2B, the apparatus 200b may further include a contact area sensor 240.

The contact area sensor 240 may measure a contact area while the object comes into contact with the pulse wave measurer 110 and increases or decreases a pressing force on the pulse wave measurer 110. The contact area sensor 240 may be disposed above or below the pulse wave measurer 110.

The processor 120 may obtain pressure based on the contact force, measured by the force sensor 130, and the contact area measured by the contact area sensor 240.

Referring back to FIGS. 2A and 2B, upon receiving a request for estimating blood pressure from a user, the processor 120 may guide a contact state for the user. For example, upon receiving the request for estimating blood pressure, the processor 120 may obtain, from the storage 230, a reference pressure to be applied by the object to the pulse wave measurer 110, and may display the obtained reference pressure through the output interface 220 to guide the user on the pressure. Further, while the pulse wave signal is measured by the force sensor 130 in real time, the processor 120 may guide the user in real time on the measured force and/or pressure.

The communication interface 210 may communicate with an external device under the control of the processor 120 by using communication techniques, and may receive the pulse wave signal from the external device. In this case, the external device is not specifically limited, but may be various types of devices, such as a smartphone, a tablet PC, a wearable device, a cuff-type blood pressure measuring device, and the like, which may directly measure an oscillometric pulse wave signal from a user, and may manage the measured oscillometric pulse wave signal. In addition, the communication interface 210 may transmit processing results of the processor 120 to the external device.

In this case, examples of the communication techniques may include Bluetooth communication, Bluetooth Low Energy (BLE) communication, Near Field Communication (NFC), WLAN communication, Zigbee communication, Infrared Data Association (IrDA) communication, Wi-Fi Direct (WFD) communication, Ultra-Wideband (UWB) communication, Ant+ communication, WIFI communication, and mobile communication, but the communication techniques are not limited thereto.

In the case where the apparatus 200a or 200b for estimating blood pressure includes both the pulse wave measurer 110 and the communication interface 120, the processor 120 may selectively control the pulse wave measurer 110 and the communication interface 210 to obtain the pulse wave signal. In another example embodiment, the pulse wave measurer 110 may be omitted depending on characteristics of the apparatus 200a or 200b for estimating blood pressure.

The processor 120 may derive a second-order differential signal of the pulse wave signal, and may estimate blood pressure by using the second-order differential signal. In this case, the processor 120 may detect peaks and valleys from the pulse wave signal, and may estimate blood pressure based on differential values at positions, corresponding to the peaks and the valleys in the second-order differential signal, and pressure exerted between the pulse wave measurer 110 and the object. For example, as described above, the processor 120 may estimate systolic blood pressure and diastolic blood pressure based on pressure at a point, corresponding to a minimum value among the first differential values at the peaks, and pressure at a point, corresponding to a maximum value among the second differential values at the valleys, in the second-order differential signal.

The output interface 220 may output and provide the pulse wave signal, measured by the pulse wave measurer 110, and processing results of the processor 120 to the user. The output interface 220 may provide the information by various visual and/or non-visual methods using a display module, a speaker, a haptic device, and the like which are mounted in the apparatus 200a or 200b for estimating blood pressure.

For example, the output interface 220 may output the waveform of the oscillometric pulse wave signal and/or the waveform of the second-order differential signal in the form of graphs. Further, the output interface 220 may display a marker visually representing the peak and valley, the minimum value at the peak and the maximum value at the valley of the waveform of the second-order differential signal on the graph of the waveform of the pulse wave signal. Further, the output interface 220 may visually display an estimated blood pressure of a user by using various visual methods, such as by changing color, line thickness, font, and the like based on whether the estimated blood pressure value falls within or outside a normal range. Alternatively, the output interface 220 may output the estimated blood pressure by voice, or may output the estimated blood pressure using non-visual methods by providing different vibrations or tactile sensations and the like according to abnormal blood pressure levels. In addition, upon comparing the estimated blood pressure value with a previous estimation history, if it is determined that the estimated blood pressure is abnormal, the output interface 220 may provide a warning message or an alarm signal, as well as guide information on a user's action such as food information that the user should be careful about (e.g., food to avoid), related hospital information, and the like.

The storage 230 may store a variety of reference information to be used for estimating blood pressure, the obtained pulse wave signal, the estimated blood pressure value, and the like. In this case, the reference information may include user information, such as a user's age, sex, occupation, current health condition, and the like, information on a relationship between pulse waves and blood pressure, and the like, but the reference information is not limited thereto. In this case, the storage 230 may include at least one storage medium of a flash memory type memory, a hard disk type memory, a multimedia card micro type memory, a card type memory (e.g., an SD memory, an XD memory, etc.), a Random Access Memory (RAM), a Static Random Access Memory (SRAM), a Read Only Memory (ROM), an Electrically Erasable Programmable Read Only Memory (EEPROM), a Programmable Read Only Memory (PROM), a magnetic memory, a magnetic disk, and an optical disk, and the like, but is not limited thereto.

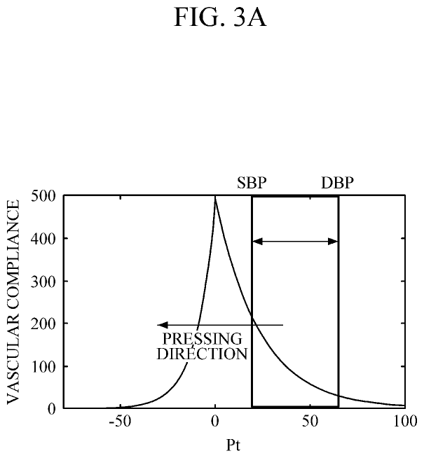

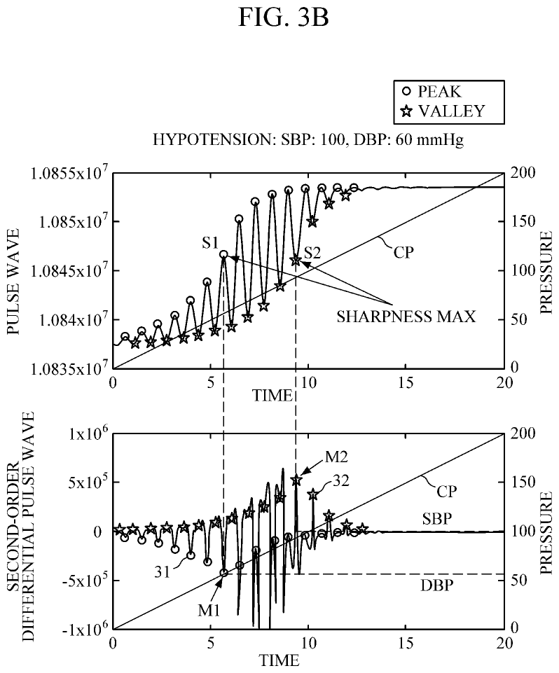

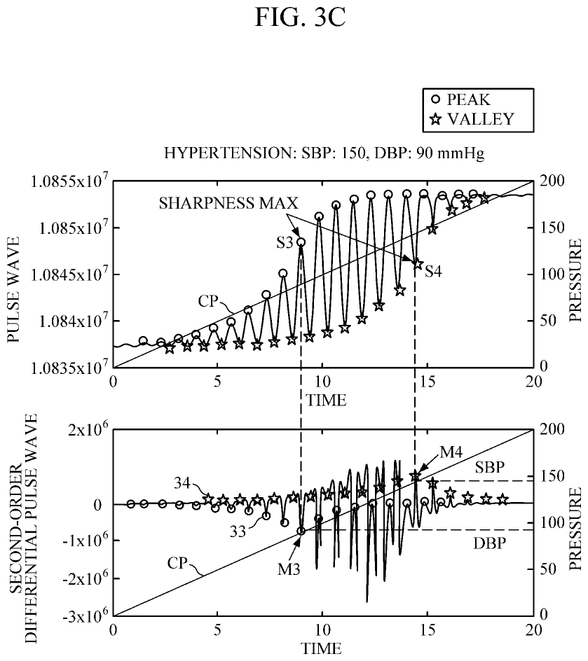

FIGS. 3A to 3C are diagrams illustrating an example of estimating systolic blood pressure and diastolic blood pressure according to an embodiment of the disclosure. An example of estimating systolic blood pressure and diastolic blood pressure from an oscillometric pulse wave signal will be described below with reference to FIGS. 1 and 3A to 3C.

FIG. 3A is a graph illustrating a relationship between transmural pressure Pt and vascular compliance, in which the relationship between the transmural pressure Pt and the vascular compliance may be obtained by differentiation.

The transmural pressure Pt may be obtained by subtracting external pressure Pe of the blood vessel from internal pressure Pi of the blood vessel. Referring to FIG. 3A, as the external pressure Pe of the blood vessel gradually increases in a pressing direction in oscillometry, there may be a point, at which transmural pressure Pt becomes zero during a systolic phase SBP or a diastolic phase DBP. In this case, as illustrated herein, the vascular compliance is maximum at the point, at which the transmural pressure Pt is zero, such that a maximum volume change of the blood vessel occurs according to a change in blood pressure, and sharpness is maximum at the peak or valley of the oscillometric pulse waves. Accordingly, by using external pressure at the position where the sharpness is maximum at the peak or valley of the pulse wave signal, systolic blood pressure or diastolic blood pressure may be estimated. In this case, the magnitude of sharpness may be obtained based on the magnitude of the absolute value of a second-order differential value of the pulse wave signal which corresponds to the peak point or the valley point.

FIG. 3B is an example of a pulse wave signal obtained from a hypotensive user, illustrating a PPG signal (upper graph), in which there is an inversely proportional relationship between pulse waves and blood pressure, a second-order differential signal (lower graph) of the PPG signal, and contact pressure CP between an object and the pulse wave measurer 110.

The processor 120 may detect peaks and valleys from the PPG signal. Further, the processor 120 may obtain a second-order differential signal, and may obtain first differential values 31, corresponding to the peaks, and second differential values 32, corresponding to the valleys, from the second-order differential signal. The processor 120 may determine a position of a minimum value M1, among the obtained first differential values 31, as a peak S1, at which sharpness is maximum among the peaks of the PPG signal. In addition, the processor 120 may determine a position of a maximum value M2, among the obtained second differential values 32, as a valley S2, at which sharpness is maximum among the valleys of the PPG signal.

Because there is an inversely proportional relationship between the PPG pulse waves and blood pressure, the processor 120 may determine pressure (about 60 mmHg) at the position of the minimum value M1 of the first differential values 31 (point where a time index is approximately 6) to be diastolic blood pressure, and may determine pressure (about 100 mmHg) at the position of the maximum value M2 of the second differential values 32 (point where a time index is approximately 9) to be systolic blood pressure.

FIG. 3C is a diagram illustrating an example of a pulse wave signal obtained from a hypertensive user, illustrating a PPG signal (upper graph), in which there is an inversely proportional relationship between pulse waves and blood pressure, a second-order differential signal (lower graph) of the PPG signal, and contact pressure CP between an object and the pulse wave measurer 110.

Likewise, the processor 120 may detect peaks and valleys from the PPG signal, and may obtain first differential values 33, corresponding to the peaks, and second differential values 34, corresponding to the valleys, from the second-order differential signal. The processor 120 may determine a position of a minimum value M3, among the obtained first differential values 31, as a peak S3, at which sharpness is maximum among the peaks of the PPG signal, and may determine a position of a maximum value M4, among the obtained second differential values 34, as a valley S4, at which sharpness is maximum among the valleys of the PPG signal.

Because there is an inversely proportional relationship between the PPG pulse waves and blood pressure, the processor 120 may determine pressure (about 90 mmHg) at the position of the minimum value M3 of the first differential value 33 (point where a time index is approximately 8) to be diastolic blood pressure, and may determine pressure (about 150 mmHg) at the position of the maximum value M4 of the second differential value 34 (point where a time index is approximately 14) to be systolic blood pressure.

The example of determining diastolic blood pressure and systolic blood pressure in the case where there is an inversely proportional relationship between the PPG pulse waves and blood pressure is described above with reference to FIGS. 3B and 3C. By contrast, in the case where there is a proportional relationship between the PPG pulse waves and blood pressure, a minimum value of a second-order differential value may be determined to be systolic blood pressure, and a maximum value of the second-order differential value at the valley may be determined to be diastolic blood pressure. As described above with reference to FIGS. 3A to 3C, the embodiments of the disclosure provide a method of obtaining systolic blood pressure and diastolic blood pressure independently of each other by using biomechanical properties of blood vessels, thereby further improving performance of estimating blood pressure based on oscillometry.

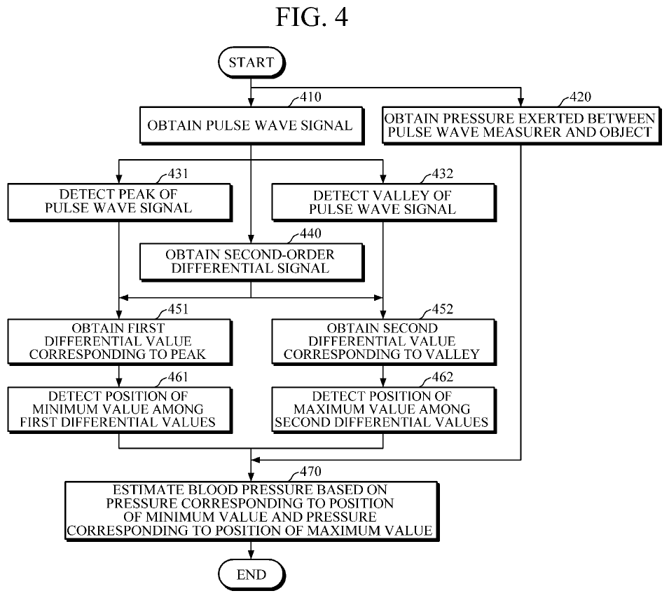

FIG. 4 is a flowchart illustrating a method of estimating blood pressure according to an embodiment of the disclosure. The method of estimating blood pressure according to the embodiment may be performed by any one of the apparatuses 100, 200a and 200b for estimating blood pressure according to the embodiments of FIGS. 1, 2A, and 2B, which are described above in detail, and thus will be briefly described below in order to avoid redundancy.

In response to a request for estimating blood pressure, the apparatus 100, 200a, or 200b for estimating blood pressure may obtain a pulse wave signal from a user's object in 410. In this case, the request for estimating blood pressure may be input by a user, may be input at predetermined blood pressure estimation intervals or may be input from an external device. In this case, examples of the pulse wave signal may include a cuff signal obtained from the upper arm and a PPG signal obtained from the wrist, finger, and the like.

Then, the apparatus 100, 200a, or 200b for estimating blood pressure may obtain pressure exerted between the pulse wave measurer and the object while the pulse wave signal is measured in 420. For example, the apparatus 100, 200a, or 200b for estimating blood pressure may include a force sensor. By using the force sensor, the apparatus 100, 200a, or 200b for estimating blood pressure may measure a force when the object changes a pressing force on the pulse wave measurer, and may obtain pressure based on the measured force. Operations 410 and 420 may not be performed in time sequence, but may be performed at the same time.

While performing operations 410 and 420, the apparatus 100, 200a, or 200b for estimating blood pressure may guide a contact state of the object. For example, upon receiving the request for estimating blood pressure, the apparatus 100, 200a, or 200b for estimating blood pressure may guide a reference contact pressure before measuring the pulse wave signal. In addition, upon obtaining the pressure between the pulse wave measurer and the object in 420, the apparatus 100, 200a, or 200b for estimating blood pressure may guide the pressure in real time while performing operations 410 and 420.

Subsequently, the apparatus 100, 200a, or 200b for estimating blood pressure may detect peaks and valleys in 431 and 432 from the pulse wave signal obtained in 410.

Next, the apparatus 100, 200a, or 200b for estimating blood pressure may obtain a second-order differential signal by performing second-order differentiation on the obtained pulse wave signal in 440, and may obtain first differential values, corresponding to the peaks, and second differential values, corresponding to the valleys, from the obtained second-order differential signal in 451 and 452.

Then, the apparatus 100, 200a, or 200b for estimating blood pressure may detect a position of a minimum value among the first differential values and a position of a maximum value among the second differential values in 461 and 462, and may estimate blood pressure based on pressure corresponding to the detected position of the minimum value and pressure corresponding to the detected position of the maximum value in 470. In this case, if there is a proportional relationship between the pulse wave signal and the blood pressure, the apparatuses 100 and 200 for estimating blood pressure may determine pressure, corresponding to the position of the minimum value among the first differential values, to be systolic blood pressure, and may determine pressure, corresponding to the position of the maximum value among the second differential values, to be diastolic blood pressure. By contrast, if there is an inversely proportional relationship between the pulse wave signal and the blood pressure, the apparatus 100, 200a, or 200b for estimating blood pressure may determine pressure, corresponding to the position of the minimum value among the first differential values, to be diastolic blood pressure, and may determine pressure, corresponding to the position of the maximum value among the second differential values, to be systolic blood pressure. Further, the apparatus 100, 200a, or 200b for estimating blood pressure may provide a blood pressure estimation result to a user by various visual and/or non-visual methods.

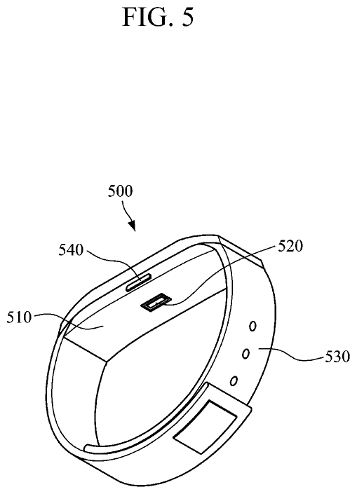

FIG. 5 is a diagram illustrating a wearable device according to an embodiment of the disclosure. One or more of the aforementioned various embodiments of the apparatuses 100, 200A, 200 for estimating blood pressure may be mounted in a smart watch wom on a wrist, but the type of the wearable device is not limited to the illustrated example.

Referring to FIG. 5, the wearable device 500 includes a main body 510 and a strap 530.

The strap 530 may be made of a flexible material. The strap 530 is connected to both ends of the main body 510, and may be wrapped around a user's wrist such that the main body 510 may be fit on the upper part of the wrist. In this case, air may be injected into the strap 530 or an airbag may be included in the strap 530, so that the strap 530 may have elasticity according to a change in pressure applied to the wrist, and the change in pressure of the wrist may be transmitted to the main body 510.

A battery may be embedded in the main body 510 or the strap 530 to supply power to various modules of the wearable device 500.

Furthermore, a pulse wave measurer 520 may be mounted on a rear surface of the main body 510. In addition, a force sensor and/or a contact area sensor may be further mounted in the main body 510. While the pulse wave measurer 520 measures the pulse wave signal on the wrist, the force sensor may measure a force applied by the upper part of the wrist to the pulse wave measurer 520. The contact area sensor may measure a contact area between an object and the pulse wave measurer 520. The pulse wave measurer 520 may include one or more light sources and detectors.

A processor may be mounted in the main body 510. The processor may estimate blood pressure by using the pulse wave signal, measured by the pulse wave measure 520, the force measured by the force sensor, and/or the contact area measured by the contact area sensor. The processor may obtain contact pressure by using the measured force, an area of the pulse wave measurer 520, or the contact area measured by the contact area sensor. As described above, the processor may estimate systolic blood pressure and diastolic blood pressure based on pressure at a position of a minimum value among differential values, corresponding to peaks, and pressure at a position of a maximum value among differential values corresponding to valleys, in the second-order differential signal of the pulse wave signal. For example, because there is generally an inversely proportional relationship between the pulse wave signal, measured on the upper portion of the wrist, and blood pressure, the processor may determine a contact pressure at a position of the minimum value, among the differential values corresponding to the peaks, to be diastolic blood pressure, and may determine a contact pressure at a position of the maximum value, among the differential values corresponding to the valleys, to be systolic blood pressure.

Further, a display may be mounted on a front surface of the main body 510, and may display guide information on a contact state or a blood pressure estimation result. In this case, the display may include a touch screen for receiving a touch input.

In addition, the main body 510 may include a storage, which stores a variety of reference information for estimating blood pressure and/or results processed by the processor.

The main body 510 may also include a manipulator 540, which is mounted on a side surface of the main body 510, and may receive a user's control command and transmit the received control command to the processor. The manipulator 540 may include a power button to input a command to turn on/off the wearable device 500. A PPG sensor may be mounted in the manipulator 540 to obtain a pulse wave signal from a finger when the finger touches the sensor.

Furthermore, a communication interface, provided for transmitting and receiving data with an external device may be mounted in the main body 510. The communication interface may communicate with an external device, e.g., a user's smartphone, a cuff-type blood pressure measuring device, and the like, to transmit and receive various data related to estimating blood pressure.

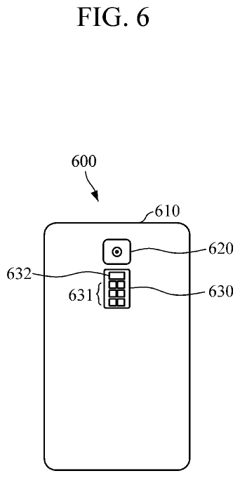

FIG. 6 is a diagram illustrating a smart device according to an embodiment of the disclosure. In this case, the smart device 600 may be a smartphone, a tablet PC, and the like, and may include the aforementioned various embodiments of the apparatuses 100, 200A, and 200B for estimating blood pressure.

Referring to FIG. 6, the smart device 600 includes a main body 610 and a pulse wave measurer 630 mounted on a rear surface of the main body 610. In this case, the pulse wave measurer 630 may include a light source 631 and a detector 632. As illustrated in FIG. 6, the pulse wave measurer 630 may be mounted on a rear surface of the main body 610, but is not limited thereto. For example, the pulse wave measurer 630 may be formed at a fingerprint sensor on a front surface of the main body 610, a portion of a touch panel, a power button and a volume button on a side surface or an upper surface of the smart device, and the like. Further, the smart device 600 may also include a force sensor and/or a contact area sensor in the main body 610.

In addition, a display may be mounted on a front surface of the main body 610. The display may display a blood pressure estimation result, guide information on a contact state, and the like.

Moreover, as illustrated in FIG. 6, an image sensor 620 may be mounted in the main body 610. When a user's finger approaches the pulse wave measurer 630 to measure a pulse wave signal, the image sensor 620 may capture an image of the finger and may transmit the captured image to the processor. In this case, based on the image of the finger, the processor may identify a relative position of the finger with respect to an actual position of the pulse wave measurer 630, and may provide the relative position of the finger to the user through the display.

The processor may estimate blood pressure by using the measured pulse wave signal and force information. As described above, by using the pulse wave signal and the second-order differential signal, the processor may estimate systolic blood pressure and diastolic blood pressure more accurately in consideration of biomechanical properties of blood vessels. A detailed description thereof will be omitted.

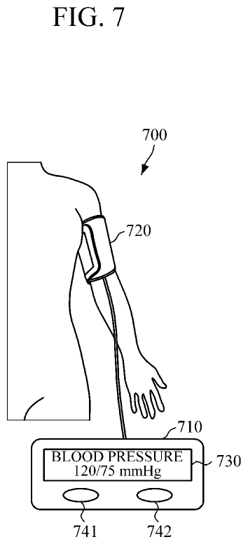

FIG. 7 is a cuff-type blood pressure measurer according to an embodiment of the disclosure.

As described above, the cuff-type blood pressure measurer 700 includes a main body 710, a cuff 720 connected to the main body 710, a display 730 mounted at the main body 710 and manipulators 741 and 742, and a processor, a communication interface and the like mounted in the main body 710.

For example, a first manipulator 741 may receive a user's request related to estimating blood pressure and may transmit the request to the processor; and a second manipulator 742 may process a user's request related to turning on/off the cuff-type blood pressure measurer 700 or communication with an external device.

In response to the request for estimating blood pressure, the processor may control the cuff 720 to obtain a cuff pulse wave and cuff pressure from a user's upper arm. Further, the processor may calculate systolic blood pressure and diastolic blood pressure from the cuff pulse wave and cuff pressure by applying the aforementioned blood pressure estimation algorithm. Because there is generally a proportional relationship between the cuff pulse wave and blood pressure obtained from an upper arm, the processor may determine a minimum cuff pressure value, among second-order differential values corresponding to peaks obtained from the cuff pulse wave, to be systolic blood pressure and may determine a maximum cuff pressure value, among second-order differential values corresponding to valleys obtained from the cuff pulse wave, to be diastolic blood pressure.

The display 730 may display an interface for a user to input various requests, including a request for estimating blood pressure, a request for communication with an external device, and the like. Further, the display 730 may display a blood pressure estimation result obtained by the processor.

The communication interface may communicate with an external device, e.g., a smart device, a wearable device, etc., and may transmit the cuff pulse wave or cuff pressure to the smart device or wearable device, so that the devices may estimate blood pressure. Alternatively, the communication interface may transmit the blood pressure estimation result of the processor to the smart device or wearable device, so that the devices may manage a user's blood pressure estimation history.

The disclosure may be implemented as a computer-readable code written on a computer-readable recording medium. The computer-readable recording medium may be any type of recording device in which data is stored in a computer-readable manner.

Examples of the computer-readable recording medium include a ROM, a RAM, a CD-ROM, a magnetic tape, a floppy disc, an optical data storage, and a carrier wave (e.g., data transmission through the Internet). The computer-readable recording medium can be distributed over a plurality of computer systems connected to a network so that a computer-readable code is written thereto and executed therefrom in a decentralized manner. Functional programs, codes, and code segments for implementing the disclosure may be easily deduced by programmers of ordinary skill in the art.

The disclosure has been described herein with regard to example embodiments. However, it will be obvious to those skilled in the art that various changes and modifications can be made without changing technical ideas and essential features of the disclosure. Thus, it is clear that the above-described embodiments are illustrative in all aspects and are not intended to limit the disclosure.

What is claimed is:

1. An apparatus for estimating blood pressure, the apparatus comprising:
a pulse wave measurer configured to measure a pulse wave signal from an object of a user; and
a processor configured to detect peaks and valleys from the pulse wave signal, to obtain first differential values, corresponding to the detected peaks, and second differential values, corresponding to the detected valleys, from a second-order differential signal of the pulse wave signal, and to estimate blood pressure based on the obtained first differential values and second differential values.

2. The apparatus of claim 1, wherein the processor is further configured to obtain pressure exerted between the object and the pulse wave measurer during measurement of the pulse wave signal.

3. The apparatus of claim 2, further comprising a force sensor configured to measure a force exerted between the pulse wave measurer and the object,
wherein the processor is further configured to obtain the pressure based on the measured force.

4. The apparatus of claim 3, further comprising a contact area sensor configured to measure a contact area between the object and the pulse wave measurer,
wherein the processor is further configured to obtain the pressure based on the measured force and the contact area.

5. The apparatus of claim 2, wherein the processor is further configured to estimate the blood pressure based on a first pressure at a position of a minimum value among the first differential values, and a second pressure at a position of a maximum value among the second differential values.

6. The apparatus of claim 5, wherein based on a relationship between a waveform of the pulse wave signal and the blood pressure, the processor is further configured to determine one of the first pressure and the second pressure to be systolic blood pressure and determine the other one of the first pressure and the second pressure to be diastolic blood pressure.

7. The apparatus of claim 6, wherein based on a proportional relationship between the waveform of the pulse wave signal and the blood pressure, the processor is further configured to determine the first pressure to be the systolic blood pressure and determine the second pressure to be the diastolic blood pressure.

8. The apparatus of claim 6, wherein based on an inversely proportional relationship between the waveform of the pulse wave signal and the blood pressure, the processor is further configured to determine the first pressure to be the diastolic blood pressure and determine the second pressure to be the systolic blood pressure.

9. The apparatus of claim 6, wherein based on at least one of the waveform of the pulse wave signal and a method of measuring the pulse wave signal, the processor is further configured to determine the relationship between the waveform of the pulse wave signal and the blood pressure.

10. The apparatus of claim 1, wherein the pulse wave measurer comprises at least one of a cuff device and a photoplethysmography (PPG) sensor.

11. The apparatus of claim 1, further comprising an output interface configured to output guide information for guiding a contact state between the object and the pulse wave measurer.

12. The apparatus of claim 1, wherein the processor is further configured to perform filtering of the measured pulse wave signal.

13. A method of estimating blood pressure, the method comprising:
measuring, by using a pulse wave measurer, a pulse wave signal from an object of a user;
detecting peaks and valleys from the pulse wave signal;
obtaining first differential values, corresponding to the detected peaks, and second differential values, corresponding to the detected valleys, from a second-order differential signal of the pulse wave signal; and
estimating blood pressure based on the obtained first differential values and second differential values.

14. The method of claim 13, further comprising obtaining pressure exerted between the object and the pulse wave measurer during measurement of the pulse wave signal.

15. The method of claim 14, wherein the obtaining the pressure comprises measuring a force exerted between the pulse wave measurer and the object, and obtaining the pressure based on the measured force.

16. The method of claim 15, wherein the obtaining the pressure further comprises measuring a contact area between the pulse wave measurer and the object, and obtaining the pressure based on the measured force and the contact area.

17. The method of claim 14, wherein the estimating the blood pressure comprises estimating the blood pressure based on a first pressure at a position of a minimum value among the first differential values, and a second pressure at a position of a maximum value among the second differential values.

18. The method of claim 17, wherein the estimating the blood pressure comprises, based on a relationship between a waveform of the pulse wave signal and blood pressure, determining one of the first pressure and the second pressure to be systolic blood pressure and determining the other one of the first pressure and the second pressure to be diastolic blood pressure.

19. The method of claim 18, wherein the estimating the blood pressure comprises, based on a proportional relationship between the waveform of the pulse wave signal and the blood pressure, determining the first pressure to be the systolic blood pressure and determining the second pressure to be the diastolic blood pressure.

20. The method of claim 18, wherein the estimating the blood pressure comprises, based on an inversely proportional relationship between the waveform of the pulse wave signal and the blood pressure, determining the first pressure to be the diastolic blood pressure and determining the second pressure to be the systolic blood pressure.

21. The method of claim 13, further comprising outputting guide information for guiding a contact state between the object and the pulse wave measurer.

22. An apparatus for estimating blood pressure, the apparatus comprising:
a communication interface configured to receive a pulse wave signal from an external device; and
a processor configured to detect peaks and valleys from the received pulse wave signal, to obtain first differential values, corresponding to the detected peaks, and second differential values, corresponding to the detected valleys, from a second-order differential signal of the pulse wave signal, and to estimate blood pressure based on the obtained first differential values and second differential values.
